(19) United States
(12) Patent Application Publication
(10) Pub. No.: US 2018/0228705 A1
(43) Pub. Date: Aug. 16, 2018

(54) ANTIPERSPIRANT

(71) Applicant: BEIERSDORF AG, Hamburg (DE)

(72) Inventors: Elke GROTHEER, Boenningstedt (DE); Franz STAEB, Echem (DE); Thomas SCHMIDT-ROSE, Hamburg (DE); Michael SEET, Mannheim (DE); Robert KLAUCK, Reinbek (DE); Frank LEHMBECK, Norderstedt (DE)

(21) Appl. No.: 15/746,842

(22) PCT Filed: Jul. 11, 2016
(86) PCT No.: PCT/EP2016/066392

§ 371 Date: Jan. 23, 2018

(30) Foreign Application Priority Data
Jul. 27, 2015 (DE) 10 2015 214 144.6

(51) Int. Cl. A61K 8/25; A61Q 15/00; A61K 8/19; A61K 8/41; B65D 83/68

(52) U.S. Cl. CPC A61K 8/25; A61Q 15/00; A61K 8/19; A61K 2800/882; B65D 83/682; A61K 2800/30; A61K 2800/87; A61K 8/41

(57) ABSTRACT

The invention relates to a cosmetic product comprising a two-chamber container for application of cosmetic preparations, characterized in that the first chamber contains a preparation comprising silicic acid (phase 1) and the second chamber a preparation comprising an alkaline compound (base) (phase 2).

The present invention relates to a cosmetic for reducing or preventing apoeccrine sweating composed of a packaging with two stock chambers and two cosmetic part preparations, characterized in that one chamber contains a part preparation comprising low-molecular-weight, highly amorphous silicic acid in combination with one or more stabilizers and the second chamber contains a preparation comprising at least one base.

Sweat denotes an aqueous secretion secreted by human skin via so-called sweat glands. There are three types of sweat glands in the skin, namely apocrine, eccrine and apoeccrine sweat glands (Int J Cosmet Sci. 2007 June; 29(3):169-79).

In humans, the eccrine sweat glands are distributed practically over the entire body and can produce considerable amounts of a clear, odorless secretion consisting of water to an extent of over 99%. By contrast, the apocrine sweat glands occur only in the hairy body areas of the underarm region and genital region and also on nipples. They produce low amounts of a milky secretion which contains proteins and lipids and is chemically neutral.

Sweating, also referred to as perspiration, is an effective mechanism of radiating excess heat and thus of regulating body temperature. What is especially used for this purpose is the high-volume aqueous secretion of the eccrine glands, which can produce up to 2-4 liters per hour, or 10-14 liters per day, in adults.

Furthermore, a signaling effect via olfaction is attributed to sweat—especially to the secretion of the apocrine sweat glands. In humans, apocrine sweat is especially important in connection with emotional or stress-related sweating.

Cosmetic antiperspirants or deodorants are used to eliminate body odor or to reduce the development thereof. Body odor develops when inherently odorless fresh sweat is decomposed by microorganisms such as, for example, staphylococci and corynebacteria.

In everyday language, there is not always a clear separation between the terms “deodorant” and “antiperspirant”. On the contrary—especially also in the German-speaking world—products for use in the underarm region are sweepingly referred to as deodorants. This is done regardless of whether there is also an antiperspirant effect.

Antiperspirants (APs) are sweat-preventing means which are intended to prevent the secretion of sweat in the first place—in contrast to deodorants, which generally prevent a microbial decomposition of sweat that has already formed.

In contrast to antiperspirants, pure deodorants do not actively influence sweat secretion, but instead merely regulate or influence body odor or underarm odor (odor improver). Different modes of action form the basis of customary cosmetic deodorants.

Common mechanisms of action in relation thereto are antibacterial effects, as also exhibited by noncolloidal silver for example, odor neutralization (masking), influencing of bacterial metabolisms, pure perfuming as well as the use of precursors of certain perfume components which are converted to fragrant substances by enzymatic reactions.

Sweat odor consists to a large extent of branched-chain fatty acids which are released from odorless sweat by bacterial enzymes. Traditional active deodorant ingredients counteract this by reducing the growth of bacteria. However, in many cases, the substances used in this connection act nonselectively even against useful skin pathogens and can lead to skin irritations in sensitive individuals.

Aluminum salts or aluminum/zirconium salts are especially used as traditional antiperspirants. They inhibit sweating by blockage of the excretory ducts of the sweat glands, by precipitating locally together with skin-endogenous proteins and thus resulting in so-called plugs. Therefore, what can occur is congestion of the sweat within the gland.

The effect of antiperspirants based on Al salts against thermal sweating under normal physiological conditions has been thoroughly investigated.

Whether this blockage is caused by denaturation of keratin or by clumping of corneocytes in the sweat gland ducts (Shelley W B and Hurley H J, Acta. Derm. Venereol. (1975) 55: 241-60), or by formation of an ACH/AZG gel (Reller H H and Luedders W L, in: Advances in Modern Toxicology, Dermatoxicology and Pharmocology, F. N. Marzulli and H. I. Maibach, Eds. Hemisphere Publishing Company, Washington and London (1977) Vol. 4: 1-5), formed by neutralization in the sweat gland duct, is still open to question.

However, the known blockage thus achieved is only effective for a short time. Heavy sweating or cleaning of the underarm as part of the normal body-washing routine cancels out the blockage and thus also the antiperspirant effect. However, the resulting necessity of applying antiperspirant (AP) products at least once daily may lead to skin irritations, especially after shaving or in or on skin areas that are already damaged.

Furthermore, aluminum salts such as aluminum chlorohydrates can cause skin damage in the event of frequent use and in sensitive individuals. Furthermore, the use of the aluminum salts can lead to discolorations of textiles which come into contact with the antiperspirant.

The additional use of antimicrobial substances in cosmetic antiperspirants can reduce the bacterial flora on the skin. In this connection, only the odor-causing microorganisms should ideally be reduced effectively. Sweating itself is not influenced thereby; ideally, only the microbial decomposition of sweat is temporarily stopped.

Customarily, antiperspirants (APs) and deodorants are provided in various product forms, with roll-ons, pump sprays and aerosols dominating in Europe and deodorant sticks being more common in the USA, Middle America and South America. Both anhydrous (suspensions) and aqueous products (aqueous/alcoholic formulations, emulsions) are known.

A satisfactory deodorant must satisfy the following requirements: 1) conservation of the natural biology of the skin 2) odor neutrality 3) efficacy only with respect to deodorization, i.e., only avoidance and/or elimination of body odor 4) avoidance of the formation of resistant bacterial strains 5) avoidance of the accumulation of the active ingredients on the skin 6) harmlessness in the event of overdosage or other unintended use 7) good cosmetic use 8) easy handling (e.g., as liquid) and universal usability in a wide variety of different cosmetic and external preparations 9) excellent skin and mucosa compatibility 10) use of environmentally friendly substances.

In addition to liquid deodorants and antiperspirants, solid preparations, for example powders and powder sprays, are also known and common as personal cleansers.

The nature of the composition of cosmetic preparations finds its natural limits which are determined by the compatibility of the individual components with one another and the stability of individual components in the carrier medium. The combination of differently charged polymers or surfactants also results in clumping and precipitation in cosmetic formulations.

There has been no lack of attempts to make such preparations accessible to the consumer. The subcomponents of the formulations are then usually packaged and stored separately. For this purpose, so-called dual chamber packaging is generally used in which the part preparations are stored in separate storage vessels and which can be withdrawn simultaneously from the package container via a common opening.

FR 2852928 discloses a dual chamber container in which the product is withdrawn by means of two pumps in such a manner that the preparations are mixed only in the output channel.

A dual chamber container is known from US 20040149775 in which the chambers are interchangeable. The preparations are also dispensed here by means of two pumps via a common output channel in which the mixing of the components takes place.

EP 958062 discloses a dual chamber packaging with pumps arranged concentrically.

FR 2900550 discloses various dual chamber packagings having a flocked applicator surface. The mixing of the components only occurs directly during application.

EP 0461010 discloses a dual chamber aerosol packaging in which the components are only mixed prior to emission from the nozzle.

Multi-chamber containers are known from US 20060054634 which function according to the bag-in-can principle and in which the at least two chambers are composed of interconnected bags.

The use of dual chamber packaging is still unknown in deodorant and/or antiperspirant products since the aluminum-containing formulations used to date, in which ACH is present as solid, are exceptionally stable.

A disadvantage of the aluminum salts used to date for sweat inhibition is the currently still incompletely explained long-term toxicity. Aluminum has been suspected for a long time of promoting or triggering neurodegenerative diseases such as dementia, especially Alzheimer's disease. In addition, aluminum is associated with the development of breast cancer. To date, there is no conclusive evidence that aluminum-containing antiperspirants acting via the skin are involved. In the case of intact skin, the maximum permitted uptake amounts cannot be reached.

However, in view of the data situation, the abandonment of aluminum-containing antiperspirants is advantageous, meaning that industry is desperately seeking aluminum-free alternatives.

On the basis of this problem, it is desirable to provide products which achieve an antiperspirant effect without use of Al salts.

One alternative to Al salts is represented by short-chain silicates in the form of silicic acids, which can reliably suppress sweating.

The oxoacids of silicon are referred to as silicic acids. The simplest silicic acid is monosilicic acid (orthosilicic acid) Si(OH)₄. It is a weak acid (pKa1=9.51; pKa2=11.74) and tends to condense. Water-elimination reactions lead to compounds such as disilicic acid (pyrosilicic acid) (HO)₃Si—O—Si(OH)₃ and trisilicic acid (HO)₃Si—O—Si(OH)₂—O—Si(OH)₃. Cyclic (annular) sillic acids are, for example, cyclotrisilicic acid and cyclotetrasilicic acid having the general empirical formula [Si(OH)₂—O—]ₙ. Polymers are occasionally referred to as metasilicic acid (H₂SO₃, [—Si(OH)₂—O—]ₙ). If these low-molecular-weight silicic acids condense further, amorphous colloids (silica sol) are formed. The general empirical formula of all silicic acids is H₂ₙ+2SiₙO₃ₙ₊₁. SiO₂.n H₂O is often specified as the empirical formula; however, in the case of silicic acid, the water is not water of crystallization, but can only be eliminated by a chemical reaction and is formed from constitutionally bound hydroxyl groups.

In general, the relatively low-water products of orthosilicic acid are covered by the term polysilicic acids. The formal end product of water elimination is silicon dioxide, the anhydride of silicic acid. The salts of the acids are called silicates. Alkali metal salts that are used or produced industrially are often called waterglasses. The esters of silicic acids are called silicic acid esters.

In the context of the invention, only silicic acids having an extent of from 1 to 100 nm, measured by dynamic light scattering, are to be understood as low-molecular-weight, highly amorphous silicic acids. Said low-molecular-weight, highly amorphous silicic acids are are also known under the designation low-molecular-weight polysilicic acids and are referred to hereinafter as LPSs.

LPSs can be produced as follows:
a) preparing an alkaline silicate solution having a pH>=10
b) lowering the pH to a pH<=1 by adding an acid, wherein the polysilicic acid is formed and wherein the pH is lowered within less than 60 seconds
c) raising the pH by addition of bases

However, the silicic acids prepared according to this method are only stable at these very low pH levels. Condensation occurs above a pH of 3.5, and this makes itself felt by precipitation gel formation. These precipitates composed of high-molecular-weight silicic acids no longer have an AP effect.

A disadvantage of the LPSs thus prepared is their incompatibility with many of the customary cosmetic auxiliaries such as emulsifiers, surfactants and oils. As a result, it is virtually impossible to produce stable customary formulation forms such as emulsions.

Since pH levels below 3.5 are physiologically incompatible, it is necessary to find preparation forms in which the pH is at least 3.5 and in which there is no gel formation or precipitation of amorphous colloids (silica sol).

Therefore, it would be desirable to provide an antiperspirant product which does not display the aforementioned disadvantages and secondary effects, more particularly ACH-containing preparations.

Furthermore, it would be desirable to provide an antiperspirant product which makes a contribution to the prior art and represents an alternative to the known preparations, more particularly ACH-containing preparations.

In particular, it is also desirable to provide an antiperspirant product which allows the broadest possible options in galenic formulation in cosmetically acceptable and attractive formula systems.

It is thus a further object of the present invention to develop a product which is suitable as a base for cosmetic deodorants or antiperspirants and do not have the disadvantages of the prior art, particularly characterized by good skin compatibility.

In particular, it is an object of the invention to provide silicic acid-containing antiperspirant preparations comprising low-molecular-weight polysilicic acids (LPSs) in combination with one or more stabilizers, having a physiologically compatible pH, with sufficient stability.

After all this, it was surprising and unforeseeable that silicic acid-containing preparations having a pH of at least 3.5, comprising LPSs and one or more stabilizers, are suitable for use as skin-compatible antiperspirants, i.e., for reducing or preventing apoeccrine sweating, if the pH is increased only shortly before application, and thereby overcoming the disadvantages of the prior art, the lack of stability of physiologically compatible LPS preparations.

It was astonishing that, by separately providing an LPS-containing preparation and a base in a dual chamber packaging, mixing the LPS-containing preparation with the base only shortly before or during dispensing and/or application, are not only outstandingly suitable for cosmetic purposes but, moreover, are more effective and milder than the use of stable compositions of the prior art.

The invention is therefore a cosmetic product comprising a dual chamber container suitable for applying cosmetic preparations, characterized in that one chamber contains a subpreparation comprising low-molecular-weight, highly amorphous silicic acid (LPS) in combination with one or more stabilizers (phase 1) and the second chamber contains a preparation comprising at least one base (phase 2), wherein during application the contents of the first and second chambers are simultaneously withdrawn from the chambers and phases 1 and 2 are mixed on dispensing or shortly before dispensing.

The invention encompasses accordingly the use of LPS as antiperspirant active ingredient, particularly in cosmetic and/or dermatological preparations that can be preferably applied topically, wherein the pH of the preparation is only adjusted to a physiologically compatible level prior to application, the pH of the applied preparation being in particular not less than 3.5.

By means of increasing the pH only prior to use, the storage stability of the LPS-containing preparation prior to use can be ensured.

The stabilizers are selected from

group A:

cis-3-hexenol (CAS 928-96-1), terpineol (CAS 8000-41-7), linalool (CAS 78-70-6), tetrahydrolinalool (CAS 78-69-3), triethyl citrate (CAS 77-93-0), 2-isobutyl-4-hydroxy-4-methyltetrahydropyran (CAS 63500-71-0), hexyl salicylate (CAS 6259-76-3), phenylethyl alcohol (CAS 60-12-8), 3-methyl-5-phenyl-1-pentanol (CAS 55066-48-3), 2,6-dimethyl-7-octen-2-ol (CAS 18479-58-8), benzyl salicylate (CAS 118-58-1), geraniol (CAS 106-24-1), citronellol (CAS 106-22-9) and ethyl linalool (CAS 10339-55-6);

group B: alcohols and diols

and group C: substances having at least three hydroxyl groups.

Particularly advantageous are stabilizers from the group consisting of linalool (CAS 78-70-6), benzyl salicylate (CAS 118-58-1), geraniol (CAS 106-24-1) and citronellol (CAS 106-22-9).

Especially advantageous stabilizers from group B are: ethanol, 2-propanol, PEG 8, triethylene glycol, methylphenylbutanol, decanediol, polyglyceryl-2 caprate, oxalic acid.

Especially advantageous stabilizers from group C are: sucrose (mannose, mannitol), glycerol, pentaerythritol, threitol, erythritol, hyaluronic acid.

The topical application of preparations—i.e., the use of cosmetic or dermatological preparations on the skin—containing LPSs in combination with one or more stabilizers selected from group A, B and/or C allows the reduction or prevention of stress sweating.

In the context of the invention, antiperspirant effect is understood to mean the possibility of reducing or preventing sweating. This means that LPSs act as sweat inhibitors and reduce sweating and thus indirectly also sweat odor.

By raising the pH of the LPS-containing preparation before dispensing and application, preparations are provided which have a high physiological compatibility.

Only the mixing of LPS-containing preparation with the pH increasing base only prior to dispensing from a dual chamber packaging makes possible the use of LPS-containing preparations as compatible antiperspirants.

Products with LPS according to the invention enable a sweat-inhibiting effect at the same order of magnitude as known and proven active antiperspirant ingredients, with the required concentration of LPSs being very much lower than when using ACH.

Raising the pH only prior to application also eradicates the disadvantages detailed, such as skin irritation due to excessively low pH levels of unstabilized silicic acids and the toxicity of aluminum compounds that is under discussion.

Products according to the invention therefore preferably comprise, besides an LPS-containing preparation, no further active antiperspirant substances or preparations, especially no aluminum salts, especially no ACH and/or AACH (activated aluminum chlorohydrate).

Furthermore, a major advantage of the products according to the invention is that, compared to the antiperspirants based on aluminum salts, no discolorations at all appear on the skin or clothing. So-called whitening does not occur, nor do the residues that can be observed in textiles resting directly on the underarm skin after repeated wearing and washing.

The stabilized silicic acids can be incorporated in a simple manner into the compositions suitable for the products according to the invention. Preferably, they are added as LPS solution to the remaining constituents of the formulations. In this connection, the proportion of the LPS solution can amount to up to 98% of the total amount of the formulation. In the simplest case, only a thickener and a perfume are added to the LPS suspension, wherein perfume is to be understood to mean a mixture comprising one or more individual substances that are olfactorily perceptible.

Silicic acids stabilized for the use according to the invention can, for example, be prepared on a laboratory scale according to the following methods:

Method I:

1. Preparing a diluted aqueous Na silicate solution having a pH>11

2. Reducing the pH from >11 to <1 by adding an appropriate amount of one or more strong acids within 5-10 sec.

3. Optionally raising the pH to a level less than 3.5 by adding one or more bases

4. Adding the stabilizer

Especially suitable for the acid used in step 2 for pH reduction are mineral acids such as hydrochloric acid, sulfuric acid or phosphoric acid, the anions of which are physiologically compatible and can be easily kept in solution. However, pH reduction can also be achieved with any other acids, provided that they a) can suitably lower the pH and b) the salts thereof, especially the sodium salt, are physiologically compatible and do not cause skin irritations. Preferably, hydrochloric acid is used for lowering pH.

Rapid pH reduction is crucial for successful LPS formation. If pH reduction is carried out too slowly, silicic acids having a very high molecular weight and having no antiperspirant activity are formed, culminating in gel formation.

Raising the pH in step 3 to a to a cosmetically acceptable level is optional and can be achieved with sodium hydroxide solution, potassium hydroxide solution or with weaker bases. Examples of usable bases are: 2-aminobutanol, 2-(2-aminoethoxy)ethanol, aminoethyl propanediol, aminomethyl propanediol, aminomethyl propanol, aminopropanediol, bis-hydroxyethyl tromethamine, butyl diethanolamine, butylethanolamine, dibutyl ethanolamine, diethanolamine, diethyl ethanolamine, diisopropanolamine, dimethylamino methylpropanol, dimethyl isopropanolamine, dimethyl MEA, ethanolamine, ethyl ethanolamine, isopropanolamine, methyl diethanolamine, methylethanolamine, triethanolamine, triisopropanolamine, tromethamine, polyethylenimine, tetrahydroxypropyl ethylenediamine, ammonia.

Raising the pH can likewise be achieved with buffer systems, which is also considered as base in the context of the invention, which systems are added either in aqueous solution or anhydrously. The buffer systems can consist of the abovementioned bases and cosmetically acceptable acids, and have a pH of from 3 to 11, preferably from 7 to 9. Examples of acids particularly suitable for buffer preparation are citric acid, lactic acid, tartaric acid, fatty acids, phosphoric acid, phosphonic acids, polyacrylic acids, succinic acid, malic acid, oxalic acid, amino acids.

Especially suitable for the base used in step 3 for pH elevation are alkali metal hydroxides, the cations of which are physiologically compatible and can be easily kept in solution. In particular, sodium and/or potassium hydroxide solution are suitable for pH elevation. Advantageously, pH elevation can be carried out in stages, wherein a concentrated base, for example 5N NaOH, is used for the first step and a less concentrated base, for example 0.5N NaOH, is used only to set the final pH.

It is advantageous to first bring the pH to pH 2 with 5N NaOH and to then increase it further to at least pH 3.5 with 0.5N NaOH.

Particularly suitable as stabilizers for these preparation methods are ethanol, glycerol, 2-propanol, PEG 8, triethylene glycol, urea, oxalic acid, hyaluronic acid, ethylhexylglycerol, pentaerythritol, threitol, erythritol, methylphenylbutanol, polyglyceryl-2 caprate, decanediol.

Particular preference is given to using glycerol and ethanol in the ratio of from 1:10 to 6:10, more particularly from 5 to 30% by weight glycerol and 30% by weight EtOH, based on the total amount of the Na silicate solution prepared in step 1.

Method H:

1. Preparing a diluted aqueous Na silicate solution having a pH>11

2. Adding at least one stabilizer

3. Reducing the pH from >11 to <1 by adding an appropriate amount of one or more strong acids within 5-10 sec.

4. Optionally raising the pH to a level less than 3.5 by adding one or more bases

The same conditions as for step 2 and step 3 of method I apply to the pH reduction (step 3) and the pH elevation (step 4).

Suitable stabilizers for this preparation method for stabilized LPSs are sucrose, mannose and/or mannitol.

Method III:

1. Preparing a diluted Na silicate solution having a pH>11, wherein the stabilizer(s) are produced at the same time as part of the solvent

2. Reducing the pH from >11 to <1 by adding an appropriate amount of one or more strong acids within 5-10 sec.

3. Optionally raising the pH to a level less than 3.5 by adding one or more bases.

The same conditions as for step 2 and step 3 of method I apply to the pH reduction (step 2) and the pH elevation (step 3).

A suitable stabilizer for this preparation method for stabilized LPSs is glycerol.

Especially at very high glycerol concentrations of above 70%, raising the pH with 0.1 to 0.5M sodium hydroxide in glycerol is advantageous, since this can prevent possible ‘pH spikes’.

In all three cases, what is crucial for LPS preparation is the exothermic pH reduction step. Said pH reduction must be done very rapidly, so that no monomer condensation can take place.

A necessary rapid, abrupt and uniform change in the pH in the batch volume requires a high stirring speed with very good mixing of the. Slow or incomplete mixing leads to shortening of stability and possibly reduction of AP performance.

On a relatively large scale, this rapid pH reduction is no longer manageable as a ‘batch’ process because of the high amount of heat to be dissipated. Therefore, rapid pH reduction is only possible industrially in a continuous method in which step 2 (method I and III) or step 3 (method II) is carried out by bringing together the reactants sodium silicate solution and acid in a flow-through reactor.

The stabilized LPS solutions obtained according to methods I to III are sufficiently stable at room temperature for use in products usable according to the invention. Stability increases with decreasing temperature, meaning that refrigerator storage (at 5 to 8 degrees Celsius) is advantageous.

Accordingly, the cosmetic products according to the invention, comprising a stabilized LPS formulation in one chamber, may be designed in the form of aerosols, i.e., preparations sprayable from dual chamber aerosol containers, dual chamber squeeze bottles or by means of a double pump device, or can be applied from dual chamber containers in the form of liquid compositions applicable by means of roll-on devices (application via a moving body, for example ball or roll). Also in the form of preparations that are applicable from normal dual chamber bottles and containers.

Also, one good method is spreading or rubbing by means of planar applicators, which are supplied from a dual chamber container, especially applicators having a flocked and/or textile surface, since their tendency to clog is low.

Preferred application forms for the antiperspirants with stabilized LPS are water-containing aerosols.

An advantage compared to aluminum chlorohydrate-containing AP sprays is that the stabilized LPSs are present in the preparation in a dissolved state and need not be resuspended by shaking prior to using the spray. The likelihood of the cans clogging is reduced as a result.

Furthermore, using the stabilized LPSs in dual chamber roll-ons and dual chamber pump sprays is possible and advantageous.

Pump sprays provide, like the aerosol sprays, a contactless application of the AP preparation to the skin. However, in the case of pump sprays, it is possible to dispense with pressure-tight containers. Dual chamber pump sprays can be designed to be metal-free, more particularly aluminum-free. For example, PE, PP or PET containers sealed with one or two metal-free spray pump(s) are advantageous, metal-free meaning that the pumped preparation does not come into contact with metallic components. Depending on whether only one pump is used or two pumps are used, the feed is effected via one or two riser tubes and the mixing of the LPS-containing preparation (phase 1) with the base (phase 2) takes place before or after the pump/pumps.

Aerosol Spray:

The stabilized LPSs (phase 1) are used in the antiperspirant formulations according to the invention preferably in an amount of from 0.1 to 10% by weight, LPS content based on the total mass of the preparation, i.e., including the propellants optionally present. Concentrations of from 0.5 to 3% by weight are especially advantageous.

Active ingredient solution denotes the sum of all constituents without the propellant, since the propellant is generally added only during filling.

AP Roll-On:

Advantageously, the selected proportion of one or more stabilizers in phase 1 can be up to 85% by weight, more particularly up to 30% by weight, based on the total mass of the preparation.

The selected proportion of SiO₂ equivalents in phase 1 is advantageously in the range from 0.1. To 6% by weight, preferably 0.5 to 3% by weight, based on the total mass of the preparation.

Pump Spray:

Advantageously, the selected proportion of one or more stabilizers in phase 1 can be up to 85% by weight, more particularly up to 30% by weight, based on the total mass of the preparation.

The selected proportion of LPS in phase 1 is advantageously in the range from 0.1 to 6% by weight, preferably 0.5 to 3% by weight, based on the total mass of the preparation.

In the context of the invention, it is also the case that the preparations comprising stabilized LPSs (phase 1) or the base (phase 2) comprise cosmetic excipients, as are customarily used in such preparations, e.g. preservatives, preservation aids, bactericides, perfumes, UV filters, antioxidants, water-soluble vitamins, minerals, suspended solid particles, antifoams, dyes, pigments having a coloring effect, thickening agents, moisturizers and/or humectants or other customary constituents of a cosmetic or dermatological formulation such as electrolytes, organic solvents, alcohols, polyols, emulsifiers, polymers, foam stabilizers or silicone derivatives.

Preference is given to preparing and using the preparations in a visually appealing transparent manner.

Advantageously, phase 2 is characterized in that it is present in the form of an aqueous or aqueous/alcoholic solution, or an anhydrous preparation.

Deodorants may also be added advantageously to phase 1 and phase 2.

Different modes of action form the basis of customary cosmetic deodorants. The use of antimicrobial substances as cosmetic deodorants can reduce the bacterial flora on the skin. In this connection, only the odor-causing microorganisms should ideally be reduced effectively. Sweating itself is not influenced thereby; ideally, only the microbial decomposition of sweat is temporarily stopped. The combination of astringents with antimicrobial substances in the same composition is common too.

All active ingredients common as deodorants can be advantageously used, for example odor-masking agents such as common perfume constituents, odor absorbers, for example the phyllosilicates described in DE 40 09 347, including in particular montmorillonite, kaolinite, illite, beidellite, nontronite, saponite, hectorite, bentonite, smectite, additionally for example zinc salts of ricinoleic acid. Antipathogens are likewise suitable for being incorporated into the preparations according to the invention. Advantageous substances are, for example, 2,4,4′-trichloro-2′-hydroxydiphenyl ether (irgasan), 1,6-bis(4-chloro-phenylbiguanido)hexane (chlorhexidine), 3,4,4′-trichlorocarbanilide, quaternary ammonium compounds, clove oil, mint oil, thyme oil, triethyl citrate, farnesol (3,7,11-trimethyl-2,6,10-dodecatrien-1-ol), ethylhexylglycerol, phenoxyethanol, piroctone olamine, caffeine and also the effective agents described in DE 37 40 186, DE 39 38 140, DE 42 04 321, DE 42 29 707, DE 42 29 737, DE 42 37 081, DE 43 09 372, DE 43 24 219. Sodium bicarbonate can be advantageously used too.

Similarly, an antimicrobial silver citrate complex, as described in DE 202008014407, can preferably be used as deodorizing constituent in conjunction with LPSs.

Phase 1 and/or phase 2 preferably also comprise polymers. The polymers preferably originate from the field of the celluloses and/or the polystyrenes. Advantageously, they have been hydrophobically or hydrophilically modified. They serve to adjust the viscosity of the phases and facilitate the pumpability and miscibility and also the draining and distribution behavior on the skin.

Therefore, useful polymers encompass celluloses, polystyrenes and/or alkyl/acryl crosspolymers and can optionally be added to the LPS preparations.

The customary cosmetic ingredients of phases 1 and 2 of the product according to the invention, in addition to water, ethanol and isopropanol, glycerol and propylene glycol, may also be skincare lipids or lipoids and also oils, such as decyl oleate, cetyl alcohol, cetylstearyl alcohol and 2-octyldodecanol, in the proportions customary for such preparations, and also mucilaginous and film-forming substances and thickening agents, e.g. hydroxyethylcellulose or hydroxypropylcellulose, polyacrylic acid, polyvinylpyrrolidone and waxes.

From the emulsifiers known for cosmetic preparations, these have been found to be advantageous for the preparations of phase 2 usable according to the invention:

polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan trioleate, polyglyceryl-10 stearate, polyglyceryl-4 caprate, lauryl glucoside, polyglyceryl-2 dipolyhydroxylstearate, polyglyceryl-10 laurate, polyglyceryl-4 laurate, decyl glucoside, propylene glycol isostearate, glycol stearate), glyceryl isostearate), sorbitan sesquioleate, glyceryl stearate, lecithin, sorbitan oleate, sorbitan monostearate NF, sorbitan stearate, sorbitan isostearate, steareth-2, oleth-2, glyceryl laurate, ceteth-2, PEG-30 dipolyhydroxystearate, glyceryl stearate SE, sorbitan stearate (and) sucrose cocoate, PEG-4 dilaurate, PEG-8 dioleate, sorbitan laurate, PEG-40 sorbitan peroleate, laureth-4, PEG-7 glyceryl cocoate, PEG-20 almond glycerides, PEG-25 hydrogenated castor oil, stearamide MEA, glyceryl stearate+PEG-100 stearate, polysorbate 85, PEG-7 olivate, cetearyl glucoside, PEG-8 oleate, polyglyceryl-3 methylglucose distearate, PEG-10 stearate, oleth-10, oleth-10/polyoxyl 10 oleyl ether NF, ceteth-10, PEG-8 laurate, ceteareth-12, cocamide MEA, polysorbate 60 NF, polysorbate 60, PEG-40 hydrogenated castor oil, polysorbate 80, isosteareth-20, PEG-60 almond glycerides, polysorbate 80 NF, PEG-150 laurate, PEG-20 methyl glucose sesquistearate, ceteareth-20, oleth-20, steareth-20, steareth-21, ceteth-20, isoceteth-20, PEG-30 glyceryl laurate, polysorbate 20, polysorbate 20 NF, laureth-23, PEG-100 stearate, steareth-100, PEG-80 sorbitan laurate.

Preference is given to using glyceryl isostearate, glyceryl stearate, steareth-2, ceteareth-20, steareth-21, PEG-40 hydrogenated castor oil, PEG-10 stearate, isoceteth-20, isosteareth-20 and ceteareth-12.

Known as solubilizers, but usable as emulsifiers for the preparations of phase 2 usable according to the invention, PEG-40 hydrogenated castor oil, polysorbate 80, laureth-23, PEG-150 laurate and PEG-30 glyceryl laurate can additionally be preferably selected.

In addition to or instead of nonionic emulsifiers, cationic emulsifiers are also suitable for generating stable formulations with the polyquaternium polymers according to the invention. Preferred suitable cationic emulsifiers can be selected from the group consisting of cetrimonium chloride, palmitamidopropyltrimonium chloride, quaternium-87, behentrimonium chloride, distearoylethyl dimonium chloride, distearyldimonium chloride, stearamidopropyl dimethylamine and/or behentrimonium methosulfate.

It is likewise advantageous to add customary antioxidants to the preparations of phase 2 in the context of the present invention. According to the invention, favorable antioxidants which can be used are all antioxidants that are suitable or customary for cosmetic and/or dermatological uses.

The amount of antioxidants (one or more compounds) in the preparations is preferably from 0.001 to 30% by weight, particularly preferably from 0.05 to 20% by weight, more particularly from 1 to 10% by weight, based on the total weight of the preparation.

If the cosmetic or dermatological preparation of phase 2 is a solution or emulsion or dispersion, it is possible to use as solvents, consistency regulators and/or active skincare ingredients:
water or aqueous solutions
oils, such as triglycerides of capric acid or of caprylic acid and alkyl benzoate, but preferably cyclic silicone oils or readily volatile hydrocarbons;
fats, waxes and other natural and synthetic lipids, preferably esters of fatty acids with alcohols of low carbon number, for example with isopropanol, propylene glycol or glycerol, or esters of fatty alcohols with alkanoic acids of low carbon number or with fatty acids; vegetable oils such as, for example, avocado oil, cuckoo flower oil, olive oil, sunflower oil, rapeseed oil, almond oil, evening primrose oil, coconut oil, palm oil, linseed oil, shea butter.
alcohols, diols or polyols of low carbon number, and also the ethers thereof, more particularly propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.
skincare substances such as, for example, panthenol, allantoin, urea, urea derivatives, guanidine, ascorbic acid, glycerylglucose.

In particular, mixtures of the aforementioned ingredients are used. In the case of alcoholic solvents, water can be a further constituent.

Suitable as propellant for cosmetic and/or dermatological preparations in the context of the present invention that are sprayable from aerosol containers are the readily volatile, liquefied propellants that are customary and known, for example hydrocarbons (propane, butane, isobutane), which propellants can be used alone or in a mixture with one another. Dimethyl ether, nitrous oxide, carbon dioxide, nitrogen and compressed air can be advantageously used too.

A person skilled in the art is of course aware that there are inherently nontoxic propellants which would be suitable in principle for the realization of the present invention in the form of aerosol preparations, but which should nevertheless be dispensed with because of environmental unfriendliness or other concomitants, especially fluorinated hydrocarbons and chlorofluorocarbons (CFCs).

In the case of aerosol preparations, oils miscible in the active-ingredient solution with the propellant (propane, butane, isobutane) are added in many cases, since an oil which is immiscible leads to precipitates, which in a glass aerosol container result in it no longer being possible to shake up the active-ingredient particles.

Cosmetic preparations of phase 2 can also be present as gels containing not only an effective content of the active ingredient according to the invention and solvents customarily used therefor, preferably water, but also organic thickening agents (thickeners), for example tamarind flour, konjac mannan, guar gum, hydroxypropyl guar, locust bean gum flour, gum arabic, xanthan gum, sodium alginate, cellulose derivatives, preferably methylcellulose, hydroxymethylcellulose, hydroxyethylcellulose, hydroxypropylcellulose, hydroxypropylmethylcellulose, carboxymethylcellulose or a mixture of polyethylene glycol and polyethylene glycol stearate or distearate. For example, the thickening agent is present in the formulation in an amount between 0.1 and 40% by weight, preferably between 0.5 and 25% by weight.

Apart from that, the customary measures for putting together cosmetic formulations must be observed, which measures are familiar to a person skilled in the art.

What follow are advantageous exemplary embodiments of the present invention.

Subsequent examples elucidate the preparations according to the invention, which for reducing or preventing sweating.

The numerical data are proportions by weight, based on the total mass of the preparation.

| Phase 1 | % by weight | % by weight | % by weight |
|---|---|---|---|
| (LPS solution with pH = 1) | A | B | C |
| Sodium silicate | 11.1 | 11.12 | 11.12 |
| Water, demineralized | 54.5 | 53.38 | 53.98 |
| Ethanol | 30 | 30 | 30 |
| HCl 37% | 4 | 4 | 4 |

| | A | B | C |
|---|---|---|---|
| Phase 2 (base) | % by weight | % by weight | % by weight |
| Sodium hydroxide (NaOH) | 0.8 | | |
| Triethanolamine (TEA) | | 3.0 | |
| 2-Amino-2-methylpropanol | | | 1.8 |
| (AMP) | | | |
| Water, demineralized | 20 | 20 | 20 |

Phase 1 and phase 2 may be dispensed from the dual chamber packaging in various ratios. Ratios of from 10:90 to 90:10 may be preset by different pump volumes depending on the selected packaging.

Tests and Proof

In order to prove antiperspirant efficacy or sweat inhibition, the sweat-reducing effect of 0.5M silicic acid was measured gravimetrically in a sauna test design.

Sauna Test Design:

The axillary amount of sweat is determined gravimetrically by cotton wool pads being weighed out after a 15 min sweating phase in the sauna.

The test subjects (N=24, 12 female+12 male) forwent the use of aluminum-containing products for at least 14 days before the start of the test. For each underarm, 500 mg of product are applied with use of left/right randomization and encryption.

6 h after application of the test products (10% ACH aqueous and 0.5M silicic acid+30% EtOH), cotton wool pads are placed in the underarms and sweat secretion is stimulated over a period of 15 min in the sauna (75° C./30% relative air humidity).

| Ingredient | “ACH” | “Silicic acid” |
|---|---|---|
| Aluminum chlorohydrate | 10% | — |
| Silicic acid | — | 11.1% (0.5M) |
| EtOH | — | 30% |
| HCl | — | 1.7 |
| NaOH | — | 0.4 |
| H₂O | to 100% | to 100% |
| pH | 4.0 ± 0.5 | 4.0 ± 0.5 |

The relative axillary amount of sweat is normalized to the sweat base level (baseline), which was recorded under identical test conditions prior to product application (=100%). Compared with the untreated area, the relative sweat reduction for the silicic acid is 54.1%. The efficacy is thus at the level of 10% ACH (see the table).

| | Baseline | After | Rel. reduction | |
|---|---|---|---|---|
| sample | [g] | application [g] | [%] | Significance p |
| “ACH” | 0.75 ± 0.45 | 0.35 ± 0.24 | 53.3 | <0.001 |
| “Silicic | 0.74 ± 0.48 | 0.34 ± 0.29 | 54.1 | <0.001 |
| acid” | | | | |

1.-12. (canceled)

13. A cosmetic product, wherein the product comprises a dual-chamber container for application of cosmetic preparations, a first chamber of the container containing a preparation comprising silicic acid (phase 1) and a second chamber of the container containing a an alkaline compound (phase 2), and wherein the container is configured to allow the contents of the first and second chambers to be simultaneously withdrawn from the chambers, phases 1 and 2 being mixed on or shortly before dispensing thereof.

14. The cosmetic product of claim 13, wherein phase 1 has a pH of less than 2.

15. The cosmetic product of claim 13, wherein phase 1 has a pH of less than 1.5.

16. The cosmetic product of claim 13, wherein phase 2 further comprises a perfume.

17. The cosmetic product of claim 13, wherein phase 2 further comprises a thickener.

18. The cosmetic product of claim 13, wherein phase 1 comprises one or more stabilizers for silicic acid.

19. The cosmetic product of claim 18, wherein the one or more stabilizers comprise one or more of cis-3-hexenol, terpineol, linalool, tetrahydrolinalool, triethyl citrate, 2-isobutyl-4-hydroxy-4-methyltetrahydropyran, hexyl salicylate, phenyl ethyl alcohol, 3-methyl-5-phenyl-1-pentanol, 2,6-dimethyl-7-octen-2-ol, benzyl salicylate, geraniol, citronellol, and ethyl linalool.

20. The cosmetic product of claim 19, wherein the one or more stabilizers comprise one or more of linalool, benzyl salicylate, geraniol, and citronellol.

21. The cosmetic product of claim 18, wherein the one or more stabilizers comprise one or more compounds selected from alcohols and diols.

22. The cosmetic product of claim 18, wherein the one or more stabilizers comprise one or more of ethanol, 2-propanol, PEG 8, triethylene glycol, methylphenylbutanol, decanediol, polyglyceryl-2 caprate, oxalic acid.

23. The cosmetic product of claim 18, wherein the one or more stabilizers comprise one or more of substances having at least three hydroxyl groups.

24. The cosmetic product of claim 18, wherein the one or more stabilizers comprise one or more of sucrose, mannose, mannitol, glycerol, pentaerythritol, threitol, erythritol, and hyaluronic acid.

25. The cosmetic product of claim 17, wherein the thickener comprises one or more of tamarind flour, konjac mannan, guar gum, hydroxypropyl guar, locust bean gum flour, gum arabic, xanthan gum, sodium alginate, and cellulose derivatives.

26. The cosmetic product of claim 17, wherein the thickener comprises one or more of methylcellulose, hydroxymethylcellulose, hydroxyethylcellulose, hydroxypropylcellulose, hydroxypropylmethylcellulose, carboxymethylcellulose.

27. The cosmetic product of claim 17, wherein the thickener comprises a mixture of polyethylene glycol and polyethylene glycol stearate or distearate.

28. The cosmetic product of claim 13, wherein phase 1 and phase 2 comprise less than 0.1% by weight of aluminum compounds.

29. The cosmetic product of claim 13, wherein phase 1 and phase 2 comprise less than 0.05 mol/l of aluminum ions.

30. The cosmetic product of claim 13, wherein phase 1 and phase 2 are free of aluminum chlorohydrate.

31. The cosmetic product of claim 13, wherein phase 2 comprises at least one physiologically compatible and/or cosmetic oil and at least one physiologically compatible and/or cosmetic emulsifier.

32. The cosmetic product of claim 13, wherein the alkaline compound of phase 2 comprises at least one of sodium hydroxide, triethanolamine, 2-amino-2-methylpropanol.
